(19) United States
(12) Patent Application Publication
(10) Pub. No.: US 2024/0366882 A1
(43) Pub. Date: Nov. 7, 2024

(54) Syringe Rigid Needle Shield With RFID Tag and Folded Antenna

(71) Applicant: Becton Dickinson France, Le Pont-de-Claix (FR)

(72) Inventors: Alfred Leibbrand, Claix (FR); Damien Maréchal, Claix (FR)

(21) Appl. No.: 18/723,882

(22) PCT Filed: Jan. 20, 2023
(86) PCT No.: PCT/EP2023/051402

§ 371 Date: Jun. 25, 2024

(30) Foreign Application Priority Data
Jan. 21, 2022 (EP) 22305065.9

(51) Int. Cl. A61M 5/32

(52) U.S. Cl. CPC A61M 5/3204; A61M 2005/3254; A61M 2205/6054

(57) ABSTRACT

A rigid needle shield assembly (40) for covering the needle of a syringe including a rigid shield (42) having an open proximal end (44), a distal end (46), an exterior sidewall (47), and an interior sidewall (48), as well as a flexible needle sheath (52) having an open proximal end (54), a closed distal end (56), an exterior sidewall (57), and an interior sidewall (58). The rigid needle shield assembly (40) also includes an RFID tag (60) having at least a first antenna leg (65A) and a second antenna leg (65B). The flexible needle sheath (52) and the RFID tag (60) are housed within the rigid shield (42). Furthermore, the RFID tag (60) is positioned between the interior sidewall (48) of the rigid shield (42) and the exterior sidewall (57) of the flexible needle sheath (52), and the first antenna leg (65A) and the second antenna leg (65B) extend along a length of the rigid shield (42) between the interior sidewall (48) of the rigid shield (42) and the exterior sidewall (57) of the flexible needle sheath (52).

### BACKGROUND

### Field of the Disclosure

The present disclosure relates to medical injection devices (e.g., syringes) utilizing rigid needle shields (RNS) having radio frequency identification (RFID) tags and folded antennae disposed therein, including methods of manufacturing such devices.

### Description of the Related Art

Medical injection devices such as, e.g., pre-fillable or prefilled syringes usually include a hollow body or barrel forming a container for a medical product. This body includes a distal end which optionally may be provided with a needle, and a proximal end, usually provided with a flange.

There is an increasing need for individual traceability of medical containers such as medical injection devices, with such traceability extending from the manufacturing process until the final labeling, final use, and/or disposal of the medical containers.

While it has been known to utilize machine-readable identifiers such as bar codes, QR codes, and/or RFID tags in order to provide traceability of products such as, e.g., syringes, such identifiers are often accessible from the surface of the containers or other devices, making the identifiers potentially subject to damage or tampering. Additionally, the location of many existing identifiers on the devices is often not conducive to effective and efficient scanning/reading.

### SUMMARY

In view of the foregoing, there exists a need for traceable identifiers such as RFID tags which may be resistant to damage and/or tampering and/or provide for improved machine readability. There is also a need for tracking a medical device from the manufacturing steps to the disposal step of said medical device.

Embodiments of the present disclosure are directed to a rigid needle shield assembly for covering the needle of a syringe. The rigid needle shield assembly includes a rigid shield having an open proximal end, a distal end, an exterior sidewall, and an interior sidewall. The rigid needle shield assembly also includes a flexible needle sheath having an open proximal end, a closed distal end, an exterior sidewall, and an interior sidewall. Additionally, the rigid needle shield assembly includes an RFID tag having at least a first antenna leg and a second antenna leg, characterized in that the flexible needle sheath and the RFID tag are disposed within the rigid shield, wherein the RFID tag is positioned between the interior sidewall of the rigid shield and the exterior sidewall of the flexible needle sheath. Furthermore, the first antenna leg and the second antenna leg extend along a length of the rigid shield between the interior sidewall of the rigid shield and the exterior sidewall of the flexible needle sheath.

In some embodiments, the RFID tag includes a dipole antenna.

In some embodiments, the RFID tag includes an integrated circuit coupled to a substrate.

In some embodiments, a first antenna leg and a second antenna leg are coupled to the substrate and are bendable relative to the substrate.

In some embodiments, the integrated circuit and the substrate are positioned between the closed distal end of the flexible needle sheath and the distal end of the rigid shield.

In some embodiments, the RFID tag includes a bendable, elongated inlay formed of flexible conductive materials.

In some embodiments, the RFID tag is U-shaped and includes an integrated circuit and a pair of leg portions having two or more antennae.

In some embodiments, the RFID tag includes an integrated circuit, a substrate, and an antenna formed by a first antenna leg, a second antenna leg, a third antenna leg, and a fourth antenna leg.

In some embodiments, the flexible needle sheath is formed of an elastomeric material.

In some embodiments, the tag is at least one of an ultra-high frequency (UHF) RFID tag, a high frequency (HF) RFID tag, a high frequency-near-field communication (HF-NFC) RFID tag, a low frequency (LF) RFID tag, a Bluetooth tag, an ultra-wide-band real-time location system (RTLS), or a Wi-Fi RTLS.

In some embodiment, the tag is configured to store a unique device identifier.

In some embodiments, the unique device identifier includes at least one of a batch number, manufacturing line, test result, time stamp, expiry date, drug identification, process setting, and component batch number pertaining to contents of the syringe upon which the rigid needle shield assembly is coupled.

In some embodiments, during assembly of the rigid needle shield assembly, the flexible needle sheath is used to press the RFID tag into position between the interior sidewall of the rigid shield and the exterior sidewall of the flexible needle sheath.

In some embodiments, the pressing of the RFID tag into position between the interior sidewall of the rigid shield and the exterior sidewall of the flexible needle sheath causes the first antenna leg and the second antenna leg to bend.

In some embodiments, during assembly of the rigid needle shield assembly, the RFID tag is selected from an RFID tag packaging strip including a plurality of serially-packaged RFID tags.

Further details and advantages of the present disclosure will be understood from the following detailed description read in conjunction with the accompanying drawings.

### BRIEF DESCRIPTION OF THE DRAWINGS

FIG. 1 is an isometric view of a syringe assembly incorporating a rigid needle shield (RNS) assembly in accordance with an aspect of the present disclosure;

FIG. 2 is a side cross-sectional view of an RNS assembly incorporating an RFID tag in accordance with an aspect of the present disclosure;

FIG. 3A is an exploded cross-sectional view of the RNS assembly of FIG. 2 in a first configuration;

FIG. 3B is an exploded cross-sectional view of the RNS assembly of FIG. 2 in a second configuration;

FIG. 4 is a partial tip view of a RFID tag packaging assembly in accordance with another aspect of the present disclosure;

FIG. 5A is an isometric view of an RFID tag in accordance with an aspect of the present disclosure;

FIG. 5B is an isometric view of an RFID tag in accordance with another aspect of the present disclosure;

FIG. 5C is an isometric view of an RFID tag in accordance with another aspect of the present disclosure;

FIG. 6A is a top plan view of an RFID tag in accordance with another aspect of the present disclosure; and

FIG. 6B is an isometric view of the RFID tag of FIG. 6A disposed over a flexible needle sheath in accordance with another aspect of the present disclosure.

### DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user holding a syringe assembly in preparation or during use. Similarly, in this application, the terms “in the distal direction” and “distally” mean in the direction of the injection, and the terms “in the proximal direction” and “proximally” mean in the direction opposite the direction of injection.

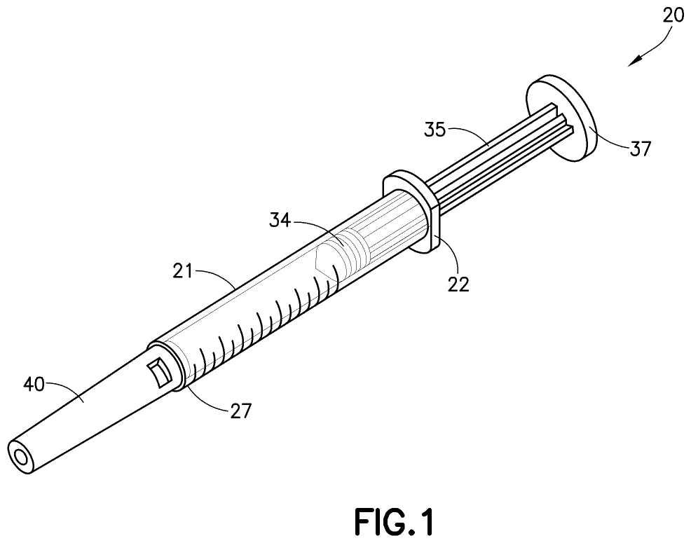

Referring to FIG. 1, a syringe assembly 20 in accordance with an aspect of the present disclosure is shown. Syringe assembly 20 includes an elongate syringe barrel 21 having a proximal end 22 and a tip portion (not shown) extending from a distal end 27 of the syringe barrel 21. The tip portion may include a sharpened needle cannula (not shown) extending therefrom, with the needle cannula being selectively coverable by a rigid needle shield (RNS) assembly 40.

A stopper 34 may be slidably positioned in fluid-tight engagement inside the syringe barrel 21, with stopper 34 engaged with a plunger rod 35 to facilitate its operation. The stopper 34 is capable of moving fluid through the syringe barrel 21 upon movement of the stopper 34 toward the distal end 27, and is also capable of drawing fluid into the syringe barrel 21 upon movement of the stopper 34 away from the distal end 27. The plunger rod 35 may be provided with a plunger rod flange 37 so as to provide structure for applying the forces for moving the plunger rod 35 (and, thus, the stopper 34) with respect to the syringe barrel 21.

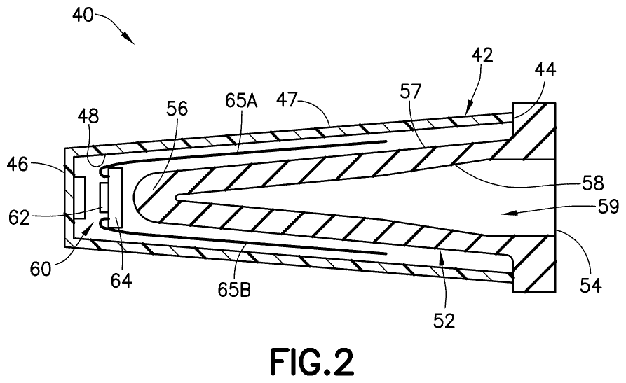

Next, referring to FIG. 2, the RNS assembly 40 in accordance with the present disclosure is shown in greater detail. RNS assembly 40 includes an exterior rigid shield 42 and an interior flexible or flexible needle sheath 52, with the flexible needle sheath 52 fixedly positioned within the rigid shield 42 after assembly. The rigid shield 42 includes an open proximal end 44 and distal end 46, with distal end 46 being at least partially closed. Rigid shield 42 also includes an exterior sidewall 47 and an interior sidewall 48. The rigid shield 42 may be formed of any appropriate rigid material such as, e.g., a rigid plastic or polymeric material (e.g., Polypropylene (PP), polyethylene (PE), polycarbonate (PC), etc.).

The flexible needle sheath 52 includes an open proximal end 54, a closed distal end 56, an exterior sidewall 57, and an interior sidewall 58. The open proximal end 54, closed distal end 56, and interior sidewall 58 define a receptacle 59 sized and configured to receive a needle of a syringe. The flexible needle sheath 52 is sized and configured to be positioned within the rigid shield 42 and, in some embodiments, may be configured such that flexible needle sheath 52 is not easily removable after production of the RNS assembly 40. The flexible needle sheath 52 may be formed of any appropriate resilient material such as, e.g., a rubber or elastomeric material such as, e.g., thermoplastic elastomers (TPEs), bromobutyl rubber, etc.

Referring still to FIG. 2, RNS assembly 40 further includes an RFID tag 60. The RFID tag 60 may be provided with unique device identifier (UDI) information, enabling each RNS assembly 40 (and, thus, each syringe) to incorporate unique, readable data regarding, e.g., batch number, manufacturing line, test results, time stamps, expiry date, drug identification, process settings, component batch number(s), etc. One or more RFID readers can then be utilized to access the UDI of each RFID tag 60, and a software and/or database solution may be provided to enable the storage and access of data linked to the UDIs of each RFID tag 60.

As shown in FIG. 2, RFID tag 60 is positioned within the interior of the rigid shield 42, disposed between the interior sidewall 48 of rigid shield 42 and the exterior sidewall 57 of the flexible needle sheath 52. In this way, the RFID tag 60 is substantially protected within the RNS assembly 40 in order to avoid damage, wear, tampering, etc., while still providing effective readability by an RFID reader or readers.

In some embodiments, RFID tag 60 may include a surface mounted integrated circuit 62 mounted to a substrate 64 (e.g., a printed circuit board (PCB), paper substrate, polymer substrate, etc.). Additionally, the RFID tag 60 may include a dipole antenna having a first antenna leg 65A and a second antenna leg 65B, with one end of each of the first antenna leg 65A and the second antenna leg 65B being coupled to the substrate 64 through any appropriate connection method such as, e.g., welding.

As shown in FIG. 2, the integrated circuit 62 and substrate 64 may be positioned between the distal end 46 of the rigid shield 42 and the distal end 56 of the flexible needle sheath 52, while the respective first antenna leg 65A and second antenna leg 65B are bent or otherwise folded such that each antenna leg extends along a length of the RNS assembly 40 between the interior sidewall 48 of rigid shield 42 and the exterior sidewall 57 of the flexible needle sheath 52. With this configuration of the first antenna leg 65A and the second antenna leg 65B, the length of the antenna of the RFID tag 60 may be maximized relative to the size of the RNS assembly 40, thereby improving the reading range of the RFID tag 60.

While first antenna leg 65A and second antenna leg 65B are only shown as extending along a portion of the RNS assembly 40, it is to be understood that each antenna leg may be longer or shorter than that which is shown in FIG. 2. For example, the first antenna leg 65A and second antenna leg 65B may be configured to each extend along substantially the entire length of the interior sidewall 48 of the rigid shield 42 in order to maximize reading range of the RFID tag 60.

Having the RFID tag 60 assembled within the RNS assembly 40 in this manner, the RFID tag 60 will inherently remain at the distal tip portion of a syringe to which the RNS assembly 40 is applied, thereby facilitating the reading of the RFID tag 60 when the syringe is nested in a tub (with or without a plurality of additional syringes also having RFID tags).

Additionally, it is to be understood that RFID tag 60 may be any appropriate RFID tag such as, e.g., an ultra-high frequency (UHF) RFID tag, a high frequency (HF) RFID tag, a high frequency—near-field communication (HF-NFC) RFID tag, and/or a low frequency (LF) RFID tag. Additionally and/or alternatively, the antenna of RFID tag 60 may be a dipole as shown in FIG. 2, or may be made of one or several loops, folded, and/or wrapped in order to increase radio frequency coupling when the RFID tag 60 is installed in the RNS assembly 40. Furthermore, while RFID tag 60 is shown and described herein, it is to be understood that other forms of remotely writable and readable data tags and/or inlays may be utilized in accordance with the present disclosure. For example, data tags or inlays such as Bluetooth tags, an ultra-wide-band real-time location system (RTLS), and/or a Wi-Fi RTLS may be utilized in lieu of (or in addition to) a RFID tag.

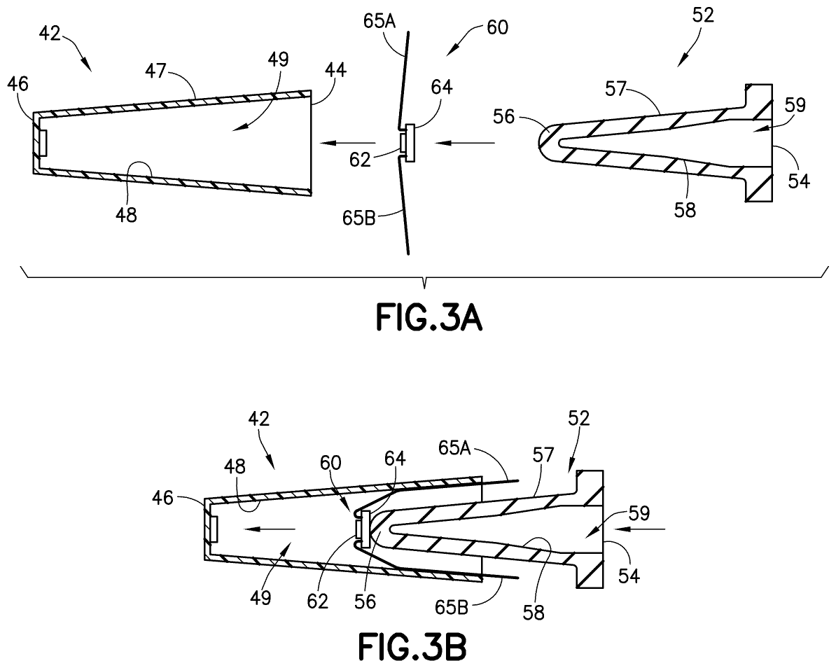

Next, referring to FIGS. 3A and 3B, a method of assembling the RNS assembly 40 in accordance with an aspect of the present disclosure is illustrated. As described above with respect to FIG. 2, the rigid shield 42, flexible needle sheath 52, and RFID tag 60 are each formed as separate elements, with both the flexible needle sheath 52 and RFID tag 60 being disposed within the rigid shield 42. In some embodiments, during production of the RNS assembly 40, the flexible needle sheath 52 may be utilized to press the RFID tag 60 into a desired position within the rigid shield 42. For example, as shown in FIG. 3A, the RFID tag 60 may be provided in a first configuration, with the first antenna leg 65A and second antenna leg 65B projecting substantially parallel to a plane formed by the substrate 64. During the manufacturing process, the RFID tag 60 may be introduced between the rigid shield 42 and the flexible needle sheath 52 such that when the flexible needle sheath 52 is pressed into an internal cavity 49 of the rigid shield 42, the RFID tag 60 is simultaneously pressed into the internal cavity 49.

Referring to FIG. 3B, as the substrate 64 of the RFID tag 60 is pressed further into the internal cavity 49 by the distal end 56 of the flexible needle sheath 52, the first antenna leg 65A and second antenna leg 65B may be caused to bend or fold inward due to their contact with the interior sidewall 48 of the rigid shield 42, ultimately being held within the RNS assembly 40 in a second configuration as shown in FIG. 2. In this way, the construction of RNS assembly 40 may remain streamlined, even with the inclusion of an integrated RFID tag 60 positioned within the interstice between the rigid shield 42 and the flexible needle sheath 52.

While not shown in FIGS. 2-3B, in other embodiments, it is to be understood that at least a portion of RFID tag 60 may be affixed or otherwise secured to the rigid shield 42 and/or the flexible needle sheath 52 by way of, e.g., an adhesive. The adhesive may be formed of any appropriate adhesive such as, e.g., a UV-cured adhesive, a rubber adhesive, a glue, a wet inlay, a silicon adhesive, a resin (e.g., an epoxy), etc. In some embodiments, the RFID tag 60 may be affixed to the outer sidewall 57 of the flexible needle sheath 52 before the insertion of the flexible needle sheath 52 into the internal cavity 49 of the rigid shield 42. In other embodiments, the RFID tag 60 may be affixed to the inner sidewall 48 of the rigid shield 42 before insertion of the flexible needle sheath 52 into the internal cavity 49 of the rigid shield.

Additionally, while the embodiment shown and described with respect to FIGS. 3A and 3B pertains to a method in which the first antenna leg 65A and second antenna leg 65B are bent during assembly of the overall RNS assembly, it is to be understood that the RFID tag 60 is not limited to such a formation. That is, the RFID tag 60 may be, e.g., pressed, folded, stamped, embossed, punched, drawn, curved, or formed to the appropriate shape to fit the interstice between the rigid shield 42 and the resilient needle shield 52 by any other appropriate method.

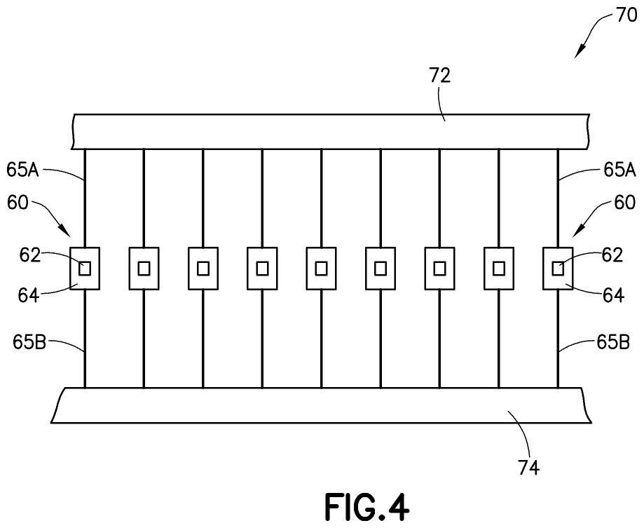

Next, referring to FIG. 4, an RFID tag packaging assembly 70 in accordance with another aspect of the present disclosure is illustrated. As noted above, production of an RNS assembly 40 including an RFID tag 60 may be automated such that the RFID tag 60 is disposed within the rigid shield 42 concurrently with the pressing of the flexible needle sheath 52 into the rigid shield 42. To further simplify the production of multiple RNS assemblies 40, a plurality of RFID tags 60 may be serially packaged relative to one another to form the RFID tag packaging strip 70 shown in FIG. 4. Specifically, distal ends of each first antenna leg 65A and second antenna leg 65B of a plurality of RFID tags 60 may be secured to respective tape portions 72, 74 of the RFID tag packaging strip 70. The RFID tag packaging strip 70 may then be provided within an RNS assembly machine, wherein each RFID tag 60 may be individually decoupled from the tape portions 72, 74 in order to be disposed within the RNS assembly 40.

While FIG. 4 illustrates an RFID tag packaging strip 70 having two separate tape portions 72, 74 securing respective ends of the first antenna leg 65A and second antenna leg 65B of a plurality of RFID tags 60, it is to be understood that only a single tape portion may suffice. Furthermore, securement of the plurality of RFID tags 60 is not limited to use with tape portions, and any other form of securement of the antenna legs, the substrate, and/or the integrated circuit(s) of the plurality of RFID tags 60 may be utilized.

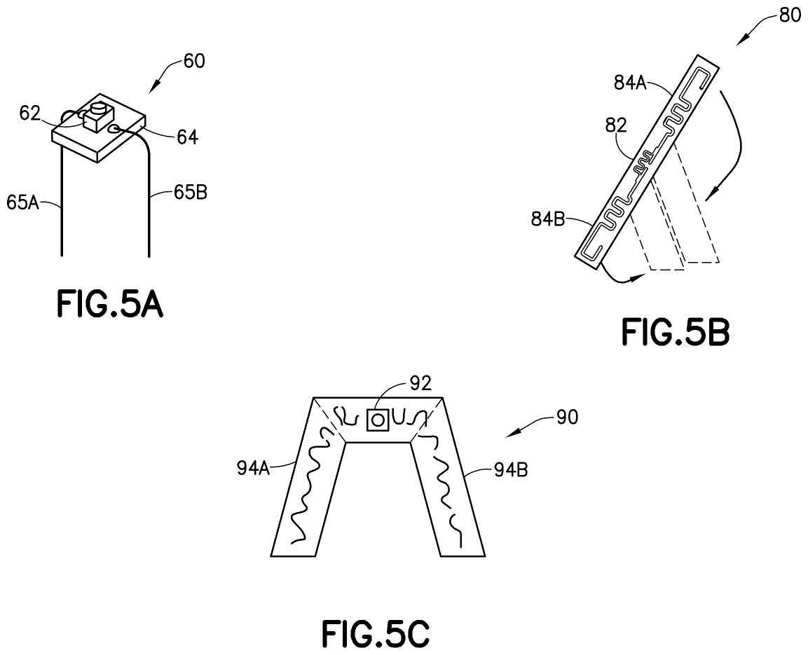

Referring now to FIGS. 5A-5C, various configurations of RFID tags in accordance with embodiments of the present disclosure are illustrated. FIG. 5A illustrates the RFID tag 60 described above with respect to FIGS. 2-4, with a surface mounted integrated circuit 62 mounted to a substrate 64, as well as a dipole antenna having a first antenna leg 65A and a second antenna leg 65B. However, other forms of RFID tag may be utilized in accordance with the present disclosure. For example, referring to FIG. 5B, an RFID tag 80 is shown, wherein RFID tag 80 is formed as an elongated and/or bendable inlay having flexible conductive materials. Such an inlay is readily available, inexpensive, and easy to manufacture. Based on the flexibility of these materials, the RFID tag 80 may be bent or folded into a shape appropriate to fit within an RNS assembly. More specifically, the RFID tag 80 may include respective leg portions 84A, 84B forming the antennae, which may be bendable relative to a central circuitry portion 82. In another embodiment, shown in FIG. 5C, an RFID tag 90 may originate as a substantially U-shaped tag, with a pair of respective leg portions 94A, 94B providing two (or more) antennas, as well as a central integrated circuit portion 82 formed between the leg portions 94A, 94B. Having a U-shaped inlay may facilitate the assembly process of the inlay, while also improving performance of the radio frequencies, thereby increasing the reading range. With these configurations, the RFID tags 80, 90 may also be disposable within an RNS assembly using a similar method as disclosed above with respect to RFID tag 60.

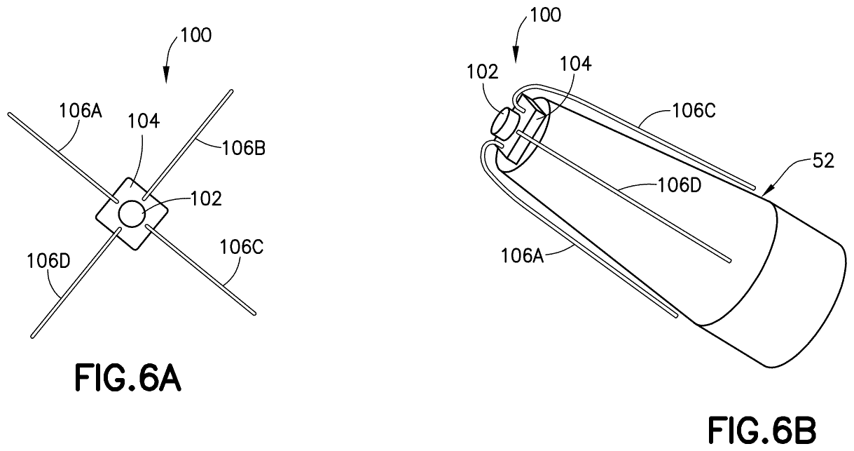

Furthermore, referring to FIGS. 6A and 6B, an RFID tag 100 in accordance with another aspect of the present disclosure is shown. The RFID tag 100 is similar to RFID tag 60 described above, but includes two additional antennae, thereby providing for improved readability of the RFID tag when housed within an RNS assembly. In particular, RFID tag 100 is configured to include a surface mounted integrated circuit 102 mounted to a substrate 104, with an antenna configuration incorporating a first antenna leg 106A, a second antenna leg 106B, a third antenna leg 106C, and a fourth antenna leg 106D. Each antenna leg 106A-106D may be coupled to the substrate 104 through any appropriate connection method such as, e.g., welding. Additionally, as shown in FIG. 6B, the antenna legs 106A-106D may be bendable/foldable around at least a portion of the flexible needle sheath 52, thereby allowing the RFID tag 100 to be secured within an RNS assembly in a manner similar to RFID tag 60 described above with respect to FIGS. 2-3B.

While several embodiments of RNS assemblies incorporating RFID tags are shown in the accompanying figures and described hereinabove in detail, other embodiments will be apparent to, and readily made by, those skilled in the art without departing from the scope and spirit of the invention. For example, it is to be understood that this disclosure contemplates, to the extent possible, that one or more features of any embodiment can be combined with one or more features of any other embodiment. Accordingly, the foregoing description is intended to be illustrative rather than restrictive.

1. A rigid needle shield assembly for covering the needle of a syringe comprising:
a rigid shield having an open proximal end, a distal end, an exterior sidewall, and an interior sidewall;
a flexible needle sheath having an open proximal end, a closed distal end, an exterior sidewall, and an interior sidewall; and
an RFID tag having at least a first antenna leg and a second antenna leg,
wherein the flexible needle sheath and the RFID tag are disposed within the rigid shield;
wherein the RFID tag is positioned between the interior sidewall of the rigid shield and the exterior sidewall of the flexible needle sheath; and
wherein the first antenna leg and the second antenna leg extend along a length of the rigid shield between the interior sidewall of the rigid shield and the exterior sidewall of the flexible needle sheath.

2. The rigid needle shield assembly of claim 1, wherein the RFID tag comprises a dipole antenna.

3. The rigid needle shield assembly of claim 1, wherein the RFID tag comprises an integrated circuit coupled to a substrate.

4. The rigid needle shield assembly of claim 3, wherein the first antenna leg and a second antenna leg are coupled to the substrate and are bendable relative to the substrate.

5. The rigid needle shield assembly of claim 3, wherein the integrated circuit and the substrate are positioned between the closed distal end of the flexible needle sheath and the distal end of the rigid shield.

6. The rigid needle shield assembly of claim 1, wherein the RFID tag comprises a bendable, elongated inlay formed of flexible conductive materials.

7. The rigid needle shield assembly of claim 1, wherein the RFID tag is U-shaped and comprises an integrated circuit and a pair of leg portions having two or more antennae.

8. The rigid needle shield assembly of claim 1, wherein the RFID tag comprises an integrated circuit, a substrate, and an antenna formed by a first antenna leg, a second antenna leg, a third antenna leg, and a fourth antenna leg.

9. The rigid needle shield assembly of claim 1, wherein the flexible needle sheath is formed of an elastomeric material.

10. The rigid needle shield assembly of claim 1, wherein the tag is at least one of an ultra-high frequency (UHF) RFID tag, a high frequency (HF) RFID tag, a high frequency—near-field communication (HF-NFC) RFID tag, a low frequency (LF) RFID tag, a Bluetooth tag, an ultra-wide-band real-time location system (RTLS), or a Wi-Fi RTLS.

11. The rigid needle shield assembly of claim 1, wherein the tag is configured to store a unique device identifier.

12. The rigid needle shield assembly of claim 11, wherein the unique device identifier includes at least one of a batch number, manufacturing line, test result, time stamp, expiry date, drug identification, process setting, and component batch number pertaining to contents of the syringe upon which the rigid needle shield assembly is coupled.

13. The rigid needle shield assembly of claim 1, wherein during assembly of the rigid needle shield assembly, the flexible needle sheath is used to press the RFID tag into position between the interior sidewall of the rigid shield and the exterior sidewall of the flexible needle sheath.

14. The rigid needle shield assembly of claim 13, wherein the pressing of the RFID tag into position between the interior sidewall of the rigid shield and the exterior sidewall of the flexible needle sheath causes the first antenna leg and the second antenna leg to bend.

15. The rigid needle shield assembly of claim 1, wherein during assembly of the rigid needle shield assembly, the RFID tag is selected from an RFID tag packaging strip comprising a plurality of serially-packaged RFID tags.
